(19) United States
(12) Patent Application Publication
(10) Pub. No.: US 2008/0032949 A1
(43) Pub. Date: Feb. 7, 2008

(54) Embedded Biocide

(76) Inventors: Christopher Jones, Cheslyn Hay (GB); Raul Diaz, Conroe, TX (US)

Correspondence Address: Jean-Louis Seugnet;RHODIA, 8 Cedar Brook Drive, CN 7500, Cranbury, NJ, 08512-7500, US

(21) Appl. No.: 10/589,210

(22) PCT Filed: Feb. 21, 2005
(86) PCT No.: PCT/GB05/00640

§ 371 Date: May 14, 2007

(30) Foreign Application Priority Data
Feb. 20, 2004 (GB) 0403773.5

(51) Int. Cl. A01N 57/00; A01P 1/00; C07C 305/00; C07C 69/00; C07F 9/02; C11D 3/36

(52) U.S. Cl. 514/75; 510/363; 558/177; 558/22; 560/1; 568/9

(57) ABSTRACT

A phosphonium compound embedded in a matrix substrate wherein the phosphonium compound is selected from a group consisting of tris (hydroxyorgano) phosphine (THP), a THP⁺ salt (tetrakis (hydroxyorgano) phosphonium salt) or a condensate of THP and a nitrogen containing compound. The compound may be used to reduce the numbers of micro organisms in industrial systems and may also be used to reduce iron carbonate or iron, lead and zinc scale deposits.

This invention relates to a phosphonium compound embedded in a matrix substrate, the use of such an embedded phosphonium compound and a method of using an embedded phosphonium compound.

Phosphonium compounds, such as THP (tris (hydroxymethyl) phosphine) and its associated salts, THP sulphate and THP chloride, are effective biocides and scale dissolvers that are widely used in water treatment and oil field applications for the control of micro-organisms and scale. Such micro-organisms include sulphate reducing bacteria, general heterotrophic bacteria and algae. These micro-organisms are responsible for scale formation in aqueous systems found in industry. Typically the scale comprises, iron carbonate or iron, lead and zinc sulphide deposits.

Phosphonium compounds are conventionally supplied as liquid based products, but solid forms of phosphonium compounds are commercially available. Solid forms typically comprise phosphonium compounds coated onto a solid, inert, substrate such as adipic acid.

Liquid based phosphonium compounds react or interfere with the performance of commonly used oxygen scavengers. For example, sulphite based scavengers and erythorbic acid, with the result being that complete deaeration of systems containing liquid based phosphonium compounds is difficult to achieve.

Accordingly, the present invention provides a phosphonium compound embedded in a matrix substrate wherein the phosphonium compound is selected from the group consisting of tris (hydroxyorgano) phosphine (THP), a THP⁺ salt (tetrakis (hydroxyorgano) phosphonium salt) or a condensate of THP and a nitrogen containing compound.

The present invention offers the following advantages:
The embedded phosphonium compound can be used for applications that neither conventional liquid or solid forms of the phosphonium compound are suitable for;
With the phosphonium compound embedded in a matrix substrate, the resulting product can be dosed into system requiring deaeration, for example, oil field water injection systems and transmission pipelines during hydrostatic testing; without impacting upon the performance of the oxygen scavenger;
A delivery system for phosphonium compounds is provided that enables the phosphonium compound to be dosed into systems containing oxygen scavengers without incompatibility problems. This is not currently possible;
A delivery system for a phosphonium compound is provided that does not adversely affect the performance of the phosphonium compound or the overall treatment program used in aqueous systems;
A delayed release system is provided that allows sustained controlled dissolution of the phosphonium compound into aqueous systems;
A delivery system is provided whereby the rates of dissolution of the embedded phosphonium compound can be controlled by the matrix composition;
A delivery system is provided that minimises direct contact with the biocide;
A delivery system is provided which is deployable in areas of low expertise and an absence of mechanical equipment, for example, pumps;
A delivery system is provided that allows phosphonium compounds to be formulated with other treatment chemicals or enhancers that would normally be incompatible if initially combined with an aqueous solution.

Preferably the THP⁺ salt is tetrakis (hydroxymethyl) phosphonium sulphate. Alternatively, the THP⁺ salt is tetrakis (hydroxymethyl) phosphonium chloride, tetrakis (hydroxymethyl) phosphonium phosphate, tetrakis (hydroxymethyl) phosphonium formate, tetrakis (hydroxymethyl) phosphonium acetate or tetrakis (hydroxymethyl) phosphonium oxalate.

The nitrogen containing compound is preferably urea. Alternatively, it may be melamine, guanidine or dicyandiamide.

The matrix substrate has a melting point of between 5 to 80° C. Preferably the melting point is between 20 to 70° C. More preferably, the melting point is 60° C.

Preferably the matrix substrate is soluble in water at a temperature of between 5 to 100° C., especially 20° C.

The matrix substrate is preferably selected from a polyhydric compound. Preferably the polyhydric compound is a polyethylene glycol with a molecular weight of above 600. More preferably, the polyhydric compound is polyethylene glycol 8000. Alternatively, the matrix substrate is selected from the group consisting of ethoxylated surfactants, fatty alcohols, ethoxylated fatty alcohols, ethoxylated alkyl phenols, ethoxylated fatty acids, fatty acid alkanolamides, ethylene oxide/propylene oxide block copolymers, ethoxylated/propoxylated fatty alcohols, polyethylene glycol esters, glycol esters, alkyl benzene sulphonic acids and salts thereof.

The matrix substrate may be a mixture of two or more of the compounds selected above.

The present invention also provides in a second aspect the use of a phosphonium compound as defined in the first aspect.

Preferably, the phosphonium compound is used to reduce the numbers of micro-organisms in industrial systems. Alternatively, the phosphonium compound is used to reduce iron carbonate or iron, lead and zinc scale deposits.

The industrial system is selected from the group consisting of storage vessels for water and fuel; fuel and gas pipelines; gas lift wells; water injection systems; oil or gas production wells; cooling tower aqueous systems; aqueous systems in paper production and the like and any other aqueous system where micro-organism contamination is a problem.

Preferably the micro-organism is selected from the group consisting of sulphate reducing bacteria, general heterotrophic bacteria and algae.

According to a third aspect, the present invention provides a method for reducing the numbers of micro-organisms in an industrial system which method comprises the step of contacting the industrial system with an effective amount of a phosphonium compound as defined in the first aspect of the invention to reduce the number of micro-organisms.

According to a fourth aspect, the present invention provides a method for reducing the amount of scale in an industrial system which method comprises the step of contacting the industrial system with an effective amount of a phosphonium compound as defined in the first aspect of the invention to reduce the amount of scale.

The phosphonium compound according to the first aspect may be formulated with one or more of the following: scale inhibitors, corrosion inhibitors, additional biocides, demulsifiers, gas hydrate inhibitors, asphaltene inhibitors/dispersants, other surfactants, anti-foams/defoamers, fragrances, wax inhibitors, scale dissolvers, gelling agents, oxygen scavengers.

The embedded biocide in accordance with the invention may be in the form of sticks/candles, beads, pellets, bricks, shavings, flakes or prills and the like.

An embodiment of the invention will now be described with reference to the following examples:

### EXAMPLE 1

A polyethylene glycol with a weight average molecular weight of 8000 (PEG8000) is used to produce matrix substrate containing THPS, that has a melting point of approximately 50° C. (suitable for storage in most areas of the world) and readily dissolving within 5 minutes in water at 20° C.

### EXAMPLE 2

As shown in Table 1 below, THPS, embedded within a PEG8000 matrix, can be deployed in the presence of an oxygen scavenger without hindering the deaeration process. Experiment three shows complete deaeration within 15 seconds when an embedded biocide in accordance with the present invention is used, compared with no deaeration when a liquid biocide is used (Experiment 2).

### EXAMPLE 3

The THPS/PEG8000 candle described above was tested in a standard quantitative suspension test to measure the antimicrobial activity of the embedded biocide. Such tests involve the addition of the biocide to the system water for a specified contact period, deactivation of the biocide after the specified contact period and subsequent enumeration of the remaining viable bacteria using Most Probable Number (MPN) techniques widely known within the industry.

From the graph below (Graph 1) it can be seen that THPS embedded in a PEG8000 matrix substrate (Tolcide® candle H2) has no adverse effects upon the antimicrobial performance of THPS when compared with liquid THPS (Tolcide® PS75).

The control shows the viability of the bacteria when not exposed to THPS.

TABLE 1

| | | Oxygen | Time to | |
|---|---|---|---|---|
| | | Scavenging | reach | |
| EXP | Description | Y/N | zero O₂ | Comments |
| 1 | 500 ppm erythorbic | Y | 15 seconds | This confirms |
| | acid (oxygen | | | the deaeration |
| | scavenger) in water: | | | efficacy of |
| | using15 ppm | | | erythrobic |
| | catalyst-CuSO₄ | | | acid- |
| 2 | Repeat of | N | Virtually no | |
| | experiment 1 but | | deaeration | |
| | 450 ppm THPS was | | occurred | |
| | introduced via a | | even after | |
| | conventional | | 15 minutes) | |
| | liquid product | | | |
| 3 | Repeat of | Y | 15 seconds | Solid |
| | experiment 1 but | | | dissolved |
| | 450 ppm THPS, | | | in ~5 min |
| | encapsulated | | | |
| | within a PEG8000 | | | |
| | candle, was | | | |
| | introduced. | | | |

1-28. (canceled)

29. A phosphonium compound embedded in a matrix substrate wherein the phosphonium compound is selected from a group consisting of tris (hydroxyorgano) phosphine (THP), a THP+ salt (tetrakis (hydroxyorgano) phosphonium salt) or a condensate of THP and a nitrogen containing compound, and wherein the matrix substrate has a melting point of between 5 to 80° C. and is soluble in water at a temperature of between 5 to 100° C. optionally of 20° C.

30. The phosphonium compound as claimed in claim 29, wherein the THP+ salt is tetrakis (hydroxymethyl) phosphonium sulphate.

31. The phosphonium compound as claimed in claim 29, wherein the THP salt is selected from the group consisting of tetrakis (hydroxymethyl) phosphonium chloride, tetrakis (hydroxymethyl) phosphonium phosphate, tetrakis (hydroxymethyl) phosphonium formate, tetrakis (hydroxymethyl) phosphonium acetate and tetrakis (hydroxymethyl) phosphonium oxalate.

32. The phosphonium compound as claimed in claim 29, wherein the nitrogen containing compound is urea.

33. The phosphonium compound as claimed in claim 29, wherein the nitrogen containing compound is melamine, guanidine or dicyandiamide.

34. The phosphonium compound as claimed in claim 29, wherein the matrix substrate has a melting point of between 20 to 70° C., optionally of 60° C.

35. The phosphonium compound as claimed in claim 34, wherein the matrix substrate is a polyhydric compound.

36. The phosphonium compound as claimed in claim 35, wherein the polyhydric compound is a polyethylene glycol with a molecular weight of above 600.

37. The phosphonium compound as claimed in claim 35, wherein the polyhydric compound is polyethylene glycol 8000.

38. The phosphonium compound as claimed in claim 29, wherein the matrix substrate is selected from the group consisting of ethoxylated surfactants, fatty alcohols, ethoxylated fatty alcohols, ethoxylated alkyl phenols, ethoxylated fatty acids, fatty acid alkanolamides, ethylene oxide/propylene oxide block copolymers, ethoxylated/propoxylated fatty alcohols, polyethylene glycol esters, glycol esters, alkyl benzene sulphonic acids and salts thereof.

39. The phosphonium compound as claimed in claim 29, wherein the matrix substrate is a mixture of two or more of the polyhydric compound as defined in claim 29.

40. A method for reducing the numbers of micro-organisms in an industrial system which method comprises the step of contacting the industrial system with an effective amount of phosphonium compound as defined in claim 29 to reduce the number of micro-organisms.

41. A method for reducing the amount of scale in an industrial system which method comprises the step of contacting the industrial system with an effective amount of a phosphonium compound as defined in claim 29 to reduce the amount of scale.

42. A formulation comprising a phosphonium compound as defined in claim 29 and one or more of the following: scale inhibitors, corrosion inhibitors, additional biocides, demulsifiers, gas hydrate inhibitors, asphaltene inhibitors/dispersants, other surfactants, anti-foams/defoamers, fragrances, wax inhibitors, scale dissolvers, gelling agents, oxygen scavengers.

43. Sticks/candles, beads, pellets, bricks, shavings, flakes or prills comprising a phosphonium compound as defined in claim 29.
